(19) United States
(12) Patent Application Publication
(10) Pub. No.: US 2004/0171703 A1
(43) Pub. Date: Sep. 2, 2004

(54) Fischer-tropsch process

(76) Inventors: Barry Nay, Surrey (GB); Christopher Sharp, East Yorkshire (GB)

Correspondence Address: NIXON & VANDERHYE, PC, 1100 N GLEBE ROAD, 8TH FLOOR, ARLINGTON, VA, 22201-4714, US

(21) Appl. No.: 10/476,229

(22) PCT Filed: May 17, 2002
(86) PCT No.: PCT/GB02/02326

(30) Foreign Application Priority Data
May 25, 2001 (GB) 0112789.3

(51) Int. Cl. C07C 27/26

(52) U.S. Cl. 518/726

(57) ABSTRACT

A process for the conversion of synthesis gas to hydrocarbons, at least a portion of which are liquid at ambient temperature and pressure, by contacting the synthesis gas at an elevated temperature and pressure with a suspension comprising a solid particulate Fischer-Tropsch catalyst suspended in a liquid medium, which contacting takes place in a reactor system comprising at least one high shear mixing zone and a reactor vessel wherein the volume of suspension present in the high shear mixing zone(s) is substantially less than the volume of suspension present in the reactor vessel, which process comprises: mixing the suspension with synthesis gas in the high shear mixing zone(s) and dissipating kinetic energy to the suspension present in the high shear mixing zone(s) at a rate of at least 0.5 kW/m³ relative to the total volume of suspension present in the reactor system; discharging the resulting mixture of synthesis gas and suspension from the high shear mixing zone(s) into the reactor vessel; withdrawing suspension from the reactor vessel and, at least in part, recycling the suspension to the high shear mixing zone(s); wherein the suspension which is recycled to the high shear mixing zone(s) is cooled to a temperature which is not more than 100° C. below the temperature of the suspension in the reactor vessel with the proviso that the temperature of the cooled suspension is at least 150° C.

The present invention relates to a process for the conversion of carbon monoxide and hydrogen (synthesis gas) to liquid hydrocarbon products in the presence of a Fischer-Tropsch catalyst.

In the Fischer-Tropsch reaction a gaseous mixture of carbon monoxide and hydrogen is reacted in the presence of a catalyst to give a hydrocarbon mixture having a relatively broad molecular weight distribution. This product is predominantly straight chain, saturated hydrocarbons which typically have a chain length of more than 2 carbon atoms, for example, more than 5 carbon atoms. The reaction is highly exothermic and therefore heat removal is one of the primary constraints of all Fischer-Tropsch processes. This has directed commercial processes away from fixed bed operation to slurry systems. Such slurry systems employ a suspension of catalyst particles in a liquid medium thereby allowing both the gross temperature control and the local temperature control (in the vicinity of individual catalyst particles) to be significantly improved compared with fixed bed operation.

Fischer-Tropsch processes are known which employ slurry bubble columns in which the catalyst is primarily distributed and suspended in the slurry by the energy imparted from the synthesis gas rising from the gas distribution means at the bottom of the slurry bubble column as described in, for example, U.S. Pat. No. 5,252,613.

The Fischer-Tropsch process may also be operated by passing a stream of the liquid medium through a catalyst bed to support and disperse the catalyst, as described in U.S. Pat. No. 5,776,988. In this approach the catalyst is more uniformly dispersed throughout the liquid medium allowing improvements in the operability and productivity of the process to be obtained.

We have recently found that a Fischer-Tropsch process may be operated by contacting synthesis gas with a suspension of catalyst in a liquid medium in a system comprising at least one high shear mixing zone and a reactor vessel. The suspension is passed through the high shear mixing zone(s) where synthesis gas is mixed with the suspension under conditions of high shear. The shearing forces exerted on the suspension in the high shear mixing zone(s) are sufficiently high that the synthesis gas is broken down into gas bubbles and/or irregularly shaped gas voids. Suspension having gas bubbles and/or irregularly shaped gas voids dispersed therein is discharged from the high shear mixing zone(s) into the reactor vessel where the majority of the conversion of synthesis gas to takes place. In the reactor vessel, mixing is aided through the action of the gas bubbles and/or the irregularly shaped gas voids on the suspension. Indeed, the suspension present in the reactor vessel is under such highly turbulent motion that any irregularly shaped gas voids are constantly coalescing and fragmenting on a rapid time scale, for example, over a time frame of up to 500 milliseconds, typically between 10 to 500 milliseconds. The transient nature of these irregularly shaped gas voids results in improved heat transfer and mass transfer of gas into the liquid phase of the suspension when compared with a conventional slurry bubble column reactor. This process is described in WO 0138269 (PCT patent application number GB 0004444) which is herein incorporated by reference. According to WO 0138269 (PCT patent application number GB 0004444), suspension may be withdrawn from the reactor vessel and may be at least in part recycled to the high shear mixing zone(s). The suspension which is recycled to the high shear mixing zone(s) may be cooled by being passed through a heat exchanger. Preferably, the recycled suspension is cooled to a temperature which is not more than 12° C. below the temperature of the suspension in the reactor vessel.

It has now been found that the process of WO 0138269 (PCT patent application number GB 0004444) may be operated by cooling the recycled suspension to a temperature of not more than 100° C. below the temperature of the suspension in the reactor vessel with the proviso that the temperature of the cooled suspension is at least 150° C.

The present invention therefore relates to a process for the conversion of synthesis gas to hydrocarbons, at least a portion of which are liquid at ambient temperature and pressure, by contacting the synthesis gas at an elevated temperature and pressure with a suspension comprising a solid particulate Fischer-Tropsch catalyst suspended in a liquid medium, which contacting takes place in a reactor system comprising at least one high shear mixing zone and a reactor vessel wherein the volume of suspension present in the high shear mixing zone(s) is substantially less than the volume of suspension present in the reactor vessel, which process comprises:

mixing the suspension with synthesis gas in the high shear mixing zone(s) and dissipating kinetic energy to the suspension present in the high shear mixing zone(s) at a rate of at least 0.5 kW/m³ relative to the total volume of suspension present in the reactor system;

discharging the resulting mixture of synthesis gas and suspension from the high shear mixing zone(s) into the reactor vessel;

withdrawing suspension from the reactor vessel and, at least in part, recycling the suspension to the high shear mixing zone(s);

wherein the suspension which is recycled to the high shear mixing zone(s) is cooled to a temperature which is not more than 100° C. below the temperature of the suspension in the reactor vessel with the proviso that the temperature of the cooled suspension is at least 150° C.

An advantage of the process of the present invention is that cooling the suspension recycle stream, outside of the reactor vessel, provides greater control over the temperature of the suspension in the reactor vessel and mitigates the risk of any thermal runaways. This increased control over the temperature of the suspension in the reactor vessel allows the process to be operated at optimum carbon monoxide conversions and also minimizes the production of by-products such as methane.

The suspension which is recycled to the high shear mixing zone(s) (hereinafter “suspension recycle stream”) may be cooled by passing the suspension recycle stream through a heat exchanger. It is also envisaged that additional cooling may be provided by means of an internal heat exchanger comprising cooling tubes, coils or plates positioned within the suspension in the reactor vessel.

Preferably, the temperature of the suspension in the reactor vessel is maintained at or near a value at which optimal conversion of synthesis gas to liquid hydrocarbon products occurs. Preferably, the temperature of the suspension in the reactor vessel is such that the carbon monoxide conversion is in the range 1 to 95%, more preferably 30 to 90%, most preferably at least 50%, for example, at least 65%.

Preferably, the temperature of the suspension in the reactor vessel is maintained at a temperature in the range of 180 to 380° C., more preferably, 200 to 230° C.

Preferably, the suspension recycle stream is cooled to a temperature which is not more than 50° C. below, more preferably not more than 25° C. below, most preferably not more than 15° C. below the temperature of the suspension in the reactor vessel. Suitably, the suspension recycle stream is cooled to a temperature which is at least 1° C. below, preferably, at least 5° C. below, more preferably at least 8° C. below, for example, at least 10° C. below the temperature of the suspension in the reactor vessel. Suitably, the temperature of the cooled suspension recycle stream is at least 150° C.

Preferably, the suspension recycle stream is cooled to a temperature at which the carbon monoxide conversion is less than 10%. The temperature at which the carbon monoxide conversion is less than 10% is generally in the range 150 to 190° C.

Preferably, the time interval between cooling the suspension and recycling the cooled suspension to the high shear mixing zone(s) is in the range 1 second to 5 minutes, more preferably, 1 second to 1 minute, for example 1 second to 20 seconds.

The volume of suspension recycled to the high shear mixing zone(s) per hour will depend on the production capacity of a commercial plant, which is typically at least 30,000 barrels of liquid hydrocarbons per day. Suitably, the suspension is recycled at a rate of between 10,000 m³ per hour and 50,000 m³ per hour, preferably, 15,000 to 30,000 m³ of suspension per hour, more preferably 17,000 to 25,000 m³ of suspension per hour for a 30,000 barrel/day plant. For larger or smaller scale capacity production plants, the rate at which the suspension is recycled to the high shear mixing zone(s) will be pro rata to the size of the plant.

The high shear mixing zone(s) may be part of the system inside or outside the reactor vessel, for example, the high shear mixing zone(s) may project through the walls of the reactor vessel such that the high shear mixing zone(s) discharges its contents into the reactor vessel. Preferably, the reactor system comprises up to 250 high shear mixing zones, more preferably less than 100, most preferably less than 50, for example 10 to 50 high shear mixing zones. Preferably, the high shear mixing zone(s) discharge into or are located within a single reactor vessel as described in WO 0138269 (PCT patent application number GB 0004444). It is also envisaged that the carbon monoxide conversion may be increased by employing 2 or 3 such reactor systems in series. Preferred arrangements of the high shear mixing zone(s) inside or outside the reactor vessel are as described in WO 0138269 (PCT patent application number GB 0004444) which is herein incorporated by reference.

Preferably, the volume of suspension present in the high shear mixing zone(s) is substantially smaller than the volume of suspension present in the remainder of the reactor system. Suitably, the volume of suspension present in the high shear mixing zone(s) is less than 20%, preferably less than 10% of the total volume of suspension present in the remainder of the reactor system.

For avoidance of doubt, it is believed that the conversion of synthesis gas into hydrocarbon products is initiated in the high shear mixing zone(s). However, the majority of the conversion of synthesis gas to hydrocarbon products takes place in the reactor vessel.

Suitably, the shearing forces exerted on the suspension in the high shear mixing zone(s) are sufficiently high that at least a portion of the synthesis gas is broken down into gas bubbles and/or irregularly shaped gas voids. Suitably, the gas bubbles have diameters in the range of from 1 μm to 10 mm, preferably from 30 μm to 3000 μm, more preferably from 30 μm to 300 μm. Without wishing to be bound by any theory, it is believed that any irregularly shaped gas voids are transient in that they are coalescing and fragmenting on a rapid time scale, for example, over a period of up to 500 ms. The gas voids have a wide size distribution with smaller gas voids having an average diameter of 1 to 2 mm and larger gas voids having an average diameter of 10 to 15 mm.

Preferably, the kinetic energy dissipation rate in the high shear mixing zone(s) is in the range of from 0.5 to 25 kW/m³, relative to the total volume of suspension present in the system, more preferably from 0.5 to 10 kW/m³, most preferably from 0.5 to 5 kW/m³, and in particular, from 0.5 to 2.5 kW/m³.

Preferably, the high shear mixing zone(s) discharges the mixture of synthesis gas and suspension in a downwards direction (down-shot) or in an upwards direction (upshot) into the reactor vessel, more preferably in a downwards direction.

The high shear mixing zone(s) may comprise any device suitable for intensive mixing or dispersing of a gaseous stream in a suspension of solids in a liquid medium, for example, a rotor-stator device, an injector-mixing nozzle or a high shear pumping means.

The injector-mixing nozzle(s) can advantageously be executed as a venturi tube (c.f. “Chemical Engineers' Handbook” by J. H. Perry, 3^{rd} edition (1953), p.1285, FIG. 61), preferably an injector mixer (c.f. “Chemical Engineers' Handbook” by J H Perry, 3rd edition (1953), p 1203, FIG. 2 and “Chemical Engineers' Handbook” by R H Perry and C H Chilton 5^{th} edition (1973) p 6-15, FIG. 6-31) or most preferably as a liquid-jet ejector (c.f. “Unit Operations” by G G Brown et al, 4^{th} edition (1953), p.194, FIG. 210).

Alternatively, the injector-mixing nozzle(s) may be executed as a venturi plate. The venturi plate may be positioned transversely within a conduit wherein the conduit has an inlet for the suspension and an outlet for the mixture of suspension and synthesis gas. The venturi plate is preferably located close to the outlet of the conduit, for example, within 1 metre, preferably, within 0.5 metres of the outlet. Suspension is introduced into the conduit through the inlet at a sufficiently high pressure to pass through apertures in the venturi plate while synthesis gas is drawn into the conduit through at least one opening, preferably 2 to 5 openings, in the walls of the conduit. Preferably, the opening(s) is located in the walls of the conduit downstream of the venturi plate, preferably, immediately downstream of the venturi plate, for example, within 1 metre, preferably within 0.5 metres of the venturi plate. Suspension having gas bubbles and/or irregularly shaped gas voids dispersed therein is discharged into the reactor vessel though the outlet of the conduit.

The injector-mixing nozzle(s) may also be executed as a “gas blast” or “gas assist” nozzle where gas expansion is used to drive the nozzle (c.f. “Atomisation and Sprays” by Arthur H Lefebvre, Hemisphere Publishing Corporation, 1989). Where the injector-mixing nozzle(s) is executed as a “gas blast” or “gas assist” nozzle, the suspension of catalyst is fed to the nozzle at a sufficiently high pressure to allow the suspension to pass through the nozzle while the synthesis gas is fed to the nozzle at a sufficiently high pressure to achieve high shear mixing within the nozzle.

The high shear mixing zone(s) may also be executed as a high shear pumping means, for example, a paddle or propeller having high shear blades, located within a conduit wherein the conduit has an inlet for the suspension and an outlet for the mixture of suspension and synthesis gas. Suitably, the high shear pumping means is located close to the outlet of the conduit, for example, within 1 metre, preferably within 0.5 metres of the outlet. Synthesis gas is injected into the conduit, for example, via a sparger, located either immediately upstream or immediately downstream of the high shear pumping means, for example, within 1 metre, preferably within 0.5 metres of the high shear pumping means. Preferably, the synthesis gas is injected into the conduit immediately upstream of the high shear pumping means. Without wishing to be bound by any theory, the injected synthesis gas is broken down into gas bubbles and/or irregularly shaped gas voids by the fluid shear imparted to the suspension by the high shear pumping means. The resulting suspension containing entrained gas bubbles and/or irregularly shaped gas voids is then discharged into the reactor vessel through the outlet of the conduit.

Where the injector mixing nozzle(s) is executed as a venturi nozzle (either a venturi tube or as a venturi plate), the pressure drop of the suspension over the venturi nozzle is typically in the range of from 1 to 40 bar, preferably 2 to 15 bar, more preferably 3 to 7 bar, most preferably 3 to 4 bar. Preferably, the ratio of the volume of gas (Q_{g}) to the volume of liquid (Qₗ) passing through the venturi nozzle is in the range 0.5:1 to 10:1, more preferably 1:1 to 5:1, most preferably 1:1 to 2.5:1, for example, 1:1 to 1.5:1 (where the ratio of the volume of gas (Q_{g}) to the volume of liquid (Qₗ) is determined at the desired reaction temperature and pressure).

Where the injector mixing nozzle(s) is executed as a gas blast or gas assist nozzle, the pressure drop of gas over the nozzle is preferably in the range 3 to 100 bar and the pressure drop of suspension over the nozzle is preferably in the range of from 1 to 40 bar, preferably 4 to 15 bar, most preferably 4 to 7 bar. Preferably, the ratio of the volume of gas (Q_{g}) to the volume of liquid (Qₗ) passing through the nozzle is in the range 0.5:1 to 50:1, preferably 1:1 to 10:1 (where the ratio of the volume of gas (Q_{g}) to the volume of liquid (Qₗ) is determined at the desired reaction temperature and pressure).

Preferably, the suspension which is withdrawn from the reactor vessel is at least in part recycled to a high shear mixing zone(s) through an external conduit having a first end in communication with an outlet (for the suspension) of the reactor vessel and a second end in communication with an inlet of the high shear mixing zone(s). The suspension may be recycled to the high shear mixing zone(s) via a mechanical pumping means, for example, a slurry pump, positioned in the external conduit. The suspension recycle stream may be cooled by means of an external heat exchanger positioned on the external conduit. It is also envisaged that an internal heat exchanger comprising cooling tubes, coils or plates, may be positioned within the suspension in the reactor vessel.

Suitably, the ratio of the volume of the external conduit (excluding the volume of the external heat exchanger) to the volume of the reactor vessel is in the range of 0.005:1 to 0.2:1.

Preferably, a stream comprising a coolant liquid, for example, a low boiling hydrocarbon(s) (such as methanol, ethanol, dimethyl ether, tetrahydrofuran, pentanes, hexanes, hexenes) and/or water may be introduced into the high shear mixing zone(s) and/or the reactor vessel as described in WO 0138269 (PCT patent application number GB 0004444). The coolant liquid may also be introduced into the external conduit.

For practical reasons the reactor vessel may not be totally filled with suspension during the process of the present invention so that above a certain level of suspension a gas cap containing a gaseous phase comprising unconverted synthesis gas, carbon dioxide, inert gases such as nitrogen, gaseous hydrocarbons, vaporized low boiling liquid hydrocarbons, vaporized water by-product and any vaporized liquid coolant is present in the top of reactor vessel. Suitably, the volume of the gas cap is not more than 40%, preferably not more than 30% of the volume of the reactor vessel. The high shear mixing zone(s) may discharge into the reactor vessel either above or below the level of suspension in the reactor vessel.

Where the reactor vessel has a gas cap, a gaseous stream may be recycled from the gas cap to the high shear mixing zone(s), for example, as described in WO 0138269 (PCT patent application number GB 0004444). It is also envisaged that the reactor vessel may be fitted with an overhead condenser or cooler for removal of heat from the gases in the gas cap. Where the reactor vessel is fitted with an overhead condenser or cooler, the gaseous recycle stream may be withdrawn from the overhead condenser or cooler also as described in WO 0138269 (PCT patent application number GB 0004444).

The process of the present invention can be operated in batch or continuous mode, the latter being preferred.

Where the process of the present invention is operated in a continuous mode, it is preferred that the average residence time of the liquid component of the suspension in the system is in the range from 10 minutes to 50 hours, preferably 1 hour to 30 hours. Suitably, the gas residence time in the high shear mixing zone(s) (for example, the injector-mixing nozzle(s)) is in the range 20 milliseconds to 2 seconds, preferably 50 to 250 milliseconds. Suitably, the gas residence time in the reactor vessel is in the range 10 to 240 seconds, preferably 20 to 90 seconds. Suitably, the gas residence time in the external conduit is in the range 10 to 180 seconds, preferably 25 to 60 seconds.

Preferably, the process of the present invention is operated with a gas hourly space velocity (GHSV) in the range 100 to 40000 h⁻¹, more preferably 1000 to 30000 h⁻¹, most preferably 2000 to 15000 h⁻¹, for example, 4000 to 10000 h⁻¹ at normal temperature and pressure (NTP) based on the feed volume of synthesis gas at NTP.

Preferably, the ratio of hydrogen to carbon monoxide of the synthesis gas used in the process of the present invention is in the range of from 20:1 to 0.1:1 by volume, especially 5:1 to 1:1 by volume, typically 2:1 by volume. Additional components such as methane, carbon dioxide, water, and inert gases such as nitrogen may be present in the synthesis gas. Where necessary, the ratio of hydrogen to carbon monoxide in the unconverted synthesis gas within the reactor vessel may be adjusted by feeding additional hydrogen and/or carbon monoxide directly into the reactor vessel, for example, via a gas sparger. It is also envisaged that additional hydrogen and/or carbon monoxide may be fed into the external conduit in order to mitigate the risk of deactivating the solid particulate catalyst.

The synthesis gas may be prepared using any of the processes known in the art including partial oxidation of hydrocarbons, steam reforming, gas heated reforming, microchannel reforming (as described in, for example, U.S. Pat. No. 6,284,217 which is herein incorporated by reference), plasma reforming, autothermal reforming and any combination thereof. A discussion of a number of these synthesis gas production technologies is provided in “Hydrocarbon Processing” V78, N.4, 87-90, 92-93 (April 1999) and “Petrole et Techniques”, N. 415, 86-93 (July-August 1998). It is also envisaged that the synthesis gas may be obtained by catalytic partial oxidation of hydrocarbons in a microstructured reactor as exemplified in “IMRBET 3: Proceedings of the Third International Conference on Microreaction Technology”, Editor W Ehrfeld, Springer Verlag, 1999, pages 187-196. Alternatively, the synthesis gas may be obtained by short contact time catalytic partial oxidation of hydrocarbonaceous feedstocks as described in EP 0303438. Preferably, the synthesis gas is obtained via a “Compact Reformer” process as described in “Hydrocarbon Engineering”, 2000, 5, (5), 67-69; “Hydrocarbon Processing”, 79/9, 34 (September 2000); “Today's Refinery”, 15/8, 9 (August 2000); WO 99/02254; and WO 200023689. An advantage of the process of the present invention is that where the synthesis gas is obtained via a “Compact Reformer” process, the synthesis gas is at an elevated pressure, for example, approximately 20 bar. Accordingly, there is no requirement to lower the pressure of the synthesis gas before feeding the synthesis gas to the injector-mixing nozzle(s) thereby providing an energy efficient integrated Reforming/Fischer Tropsch process. In particular, the pressure of synthesis gas obtained via a “Compact Reformer” process is generally sufficiently high to achieve high shear mixing within a “gas blast” or “gas assist” nozzle.

Preferably, the hydrocarbons are liquid at ambient temperature and pressure (hereinafter “liquid hydrocarbon products”) and preferably comprise a mixture of hydrocarbons having a chain length of greater than 5 carbon atoms. Suitably, the liquid hydrocarbon products comprise a mixture of hydrocarbons having chain lengths of from 5 to about 90 carbon atoms. Preferably, a major amount, for example, greater than 60% by weight, of the liquid hydrocarbon products have chain lengths of from 5 to 30 carbon atoms. Suitably, the liquid medium comprises one or more of the liquid hydrocarbon products.

Owing to the exothermic nature of the Fischer-Tropsch synthesis reaction, the temperature of the recycled suspension will rapidly increase as the suspension is mixed with synthesis gas in the high shear mixing zone(s). The particulate catalyst will therefore be subjected to thermal cycling as the suspension recycled stream is cooled, for example, in the external conduit, and is subsequently re-heated as it is mixed with synthesis gas in the high shear mixing zone(s). The catalyst which may be employed in the process of the present invention is therefore any catalyst known to be active in Fischer-Tropsch synthesis and which is stable under thermal cycling conditions. Group VIII metals whether supported or unsupported are known Fischer-Tropsch catalysts. Of these iron, cobalt and ruthenium are preferred, particularly iron and cobalt, most particularly cobalt.

A preferred catalyst is supported on a support such as an elemental carbon, for example, graphite, or an inorganic oxide, preferably a refractory inorganic oxide, or any combination thereof. Preferred supports include silica, alumina, silica-alumina, the Group IVB oxides, titania (primarily in the rutile form) and zinc oxide. The supports generally have a surface area of less than about 100 m²/g, suitably less than 50 m²/g, for example, less than 25 m²/g or about 5 m²/g.

The catalytic metal is present in catalytically active amounts usually about 1-100 wt %, the upper limit being attained in the case of unsupported metal based catalysts, preferably 2-40 wt %. Promoters may be added to the catalyst and are well known in the Fischer-Tropsch catalyst art. Promoters can include ruthenium, platinum or palladium (when not the primary catalyst metal), rhenium, hafnium, cerium, lanthanum, aluminium and zirconium, and are usually present in amounts less than the primary catalytic metal (except for ruthenium which may be present in coequal amounts), but the promoter:metal ratio should be at least 1:10. Preferred promoters are rhenium and hafnium.

A particularly preferred catalyst is cobalt supported on an inorganic refractory oxide selected from the group consisting of silica, alumina, silica-alumina and zinc oxide, more preferably, zinc oxide.

Preferably, the catalyst has a particle size in the range 5 to 500 microns, more preferably 5 to 100 microns, most preferably, in the range 5 to 30 microns.

Preferably, the suspension of catalyst discharged into the reactor vessel comprises less than 40% wt of catalyst particles, more preferably 10 to 30% wt of catalyst particles, most preferably 10 to 20% wt of catalyst particles.

The process of the present invention is preferably carried out at a temperature of 180-380° C., more preferably 180-280° C., most preferably 190-240° C., for example, 200-230° C.

The process of the invention is preferably carried out at a pressure of 5-50 bar, more preferably 15-35 bar, generally 20-30 bar.

The liquid hydrocarbon products may be separated from the suspension, purified and optionally hydrocracked, all as described in WO 0138269 (PCT patent application number GB 0004444).

### EXAMPLE

This Example was designed to investigate the effect of temperature cycling on the stability of a Fischer-Tropsch catalyst

A sample of catalyst (10 g; 20% w/w cobalt on zinc oxide prepared by co-precipitation of cobalt nitrate and zinc nitrate with ammonium carbonate as described in, for example, U.S. Pat. No. 4,826,800 which is herein incorporated by reference) was reduced in a 3.5 cm outer diameter (OD) tubular reactor. The reactor was purged with nitrogen at a space velocity of 1000 h⁻¹ at atmospheric pressure and room temperature. The temperature of the reactor contents was raised at a rate of 2° C./min to 60° C. The gas feed was then switched over to air at 1000 GHSV (GHSV=gas hourly space velocity). The temperature was then raised at a rate of 1° C./min up to 250° C. and held at this temperature for 3 hours. The gas flow was then changed to nitrogen at 1000 GHSV for 6 minutes and then the feed gas was switched to carbon monoxide at 2000 GHSV and held for 3.5 hours. The feed gas was then changed back to nitrogen and the temperature ramped at 4° C./min up to a temperature of 280° C. Once at 280° C., the feed gas was switched to hydrogen at 2500 GHSV and held there for 10 hours. The reactor was then cooled to room temperature and purged with nitrogen prior to transferring the catalyst into a continuous stirred tank slurry reactor (CSTR) containing squalane (300 ml; ex Aldrich) under nitrogen purge.

The CSTR reactor was sealed and heated up to a temperature of 125° C. with a nitrogen flow of 250 ml/min. The feed gas to the reactor was then switched to synthesis gas at 8000 GHSV, the stirrer speed was increased to 700 rpm and the temperature was ramped at 2° C./min up to 130° C. The reactor was then pressurised to 20 barg at a rate of 30 bar/hour. The temperature was then ramped at 60° C./hour up to 160° C., 5° C./hour up to 175° C. and 1° C./hour up to 185° C. Automatic temperature control was then used to increase the % CO conversion. The automatic temperature control was set such that the temperature was ramped at 0.6° C./hour for up to 20% CO conversion and at 0.5° C./hour for over 20% CO conversion.

After 100 hours on stream a C₅+productivity of 243 g/litre of catalyst/hour was obtained at a temperature of 230° C., with a CO conversion of 22%.

After 136 hours on stream, the automatic temperature control was switched off and the temperature was held constant at 226° C., to allow the reaction to stabilise prior to the temperature cycling test.

At 162 hours on stream, the GHSV was lowered to 3000 h⁻¹ to increase the % CO conversion so that any effects of the temperature cycling experiment could be easily monitored.

At 182 hours on stream the temperature cycling experiment was started. The CO conversion was 29.6% and the C₅+Productivity was 119 g/litre of catalyst/hour. The reactor comprised one heating jacket, a cooling jacket and an internal cooling coil. The oil in the heating jacket was set to a temperature of 238° C. The oil in the cooling coils was set to a temperature of 195° C. Using automatic pneumatic valves, the flow of cooling medium round the cooling coil/jacket was controlled. The system was set up to expose the reactor to oil from the heating jacket for 3 minutes and then to the cool oil from the cooling coils for 20 seconds. This cycle was repeated 12 times. This resulted in the temperature of the reactor contents cycling from 227.8° C. to 217.9° C. and back to 227.80 in a cycle which lasted 3 minutes and 20 seconds.

After 12 cycles, the temperature was returned to 226° C. The % CO conversion at this temperature was 29.2% and the C₅+productivity was 116 g/litre of catalyst/hour. This experiment showed that temperature cycling had no effect on the performance of the catalyst which, within experimental error, had the same % CO conversion and C₅+productivity both before and after temperature cycling.

1. A process for the conversion of synthesis gas to hydrocarbons, at least a portion of which are liquid at ambient temperature and pressure, by contacting the synthesis gas at an elevated temperature and pressure with a suspension comprising a solid particulate Fischer-Tropsch catalyst suspended in a liquid medium, which contacting takes place in a reactor system comprising at least one high shear mixing zone and a reactor vessel wherein the volume of suspension present in the high shear mixing zone(s) is substantially less than the volume of suspension present in the reactor vessel, which process comprises:
mixing the suspension with synthesis gas in the high shear mixing zone(s) and dissipating kinetic energy to the suspension present in the high shear mixing zone(s) at a rate of at least 0.5 kW/m³ relative to the total volume of suspension present in the reactor system;
discharging the resulting mixture of synthesis gas and suspension from the high shear mixing zone(s) into the reactor vessel;
withdrawing suspension from the reactor vessel and, at least in part, recycling the suspension to the high shear mixing zone(s);
wherein the suspension which is recycled to the high shear mixing zone(s) is cooled to a temperature which is not more than 100° C. below the temperature of the suspension in the reactor vessel with the proviso that the temperature of the cooled suspension is at least 150° C.

2. A process as claimed in claim 1 wherein additional cooling is provided by means of an internal heat exchanger positioned within the suspension in the reactor vessel.

3. A process as claimed in any one of the preceding claims wherein the suspension in the reactor vessel is maintained at a temperature in the range of 190 to 240° C.

4. A process as claimed in any one of the preceding claims wherein the suspension recycle stream is cooled to a temperature which is not more than 50° C. below, preferably not more than 25° C. below, more preferably not more than 15° C. below the temperature of the suspension in the reactor vessel.

5. A process as claimed in claim 4 wherein the suspension recycle stream is cooled to a temperature which is at least 5° C. below, preferably at least 8° C. below, more preferably at least 10° C. below the temperature of the suspension in the reactor vessel.

6. A process as claimed in any one of the preceding claims wherein the temperature of the cooled suspension recycle stream is in the range 150 to 180° C.

7. A process as claimed in any one of the preceding claims wherein the time interval between cooling the suspension and recycling the cooled suspension to the high shear mixing zone(s) is in the range 1 second to 1 minute, preferably, 1 second to 20 seconds.

8. A process as claimed in any one of the preceding claims wherein the rate at which the suspension is recycled to the high shear mixing zone(s) is in the range of 10,000 to 50,000 m³/hour, preferably, 15,000 to 30,000 m³ of suspension per hour for a 30,000 barrel per day plant or is pro-rata for larger and smaller capacity plants.

9. A process as claimed in any one of the preceding claims wherein the volume of suspension present in the high shear mixing zone(s) is less than 20%, preferably less than 10% of the total volume of suspension present in the reactor vessel.

10. A process as claimed in any one of the preceding claims wherein the high shear mixing zone(s) discharge the mixture of synthesis gas and suspension in a downwards direction into the reactor vessel.

11. A process as claimed in any one of the preceding claims wherein the high shear mixing zone(s) comprises an injector-mixing nozzle.

12. A process as claimed in claim 11 wherein the injector-mixing nozzle(s) is executed as a venturi nozzle having a pressure drop of the suspension over the venturi nozzle in the range of from 1 to 40 bar, preferably 2 to 15 bar and wherein the ratio of the volume of gas (Q_{g}) to the volume of liquid (Qₗ) passing through the venturi nozzle is in the range 0.5:1 to 10:1, more preferably 1:1 to 5:1 (where the ratio of the volume of gas (Q_{g}) to the volume of liquid (Qₗ) is determined at the desired reaction temperature and pressure).

13. A process as claimed in claim 11 wherein the injector mixing nozzle(s) is executed as a gas blast nozzle having a pressure drop of gas over the nozzle in the range 3 to 100 bar and a pressure drop of suspension over the nozzle in the range of from 1 to 40 bar, preferably 4 to 15 bar and wherein the ratio of the volume of gas (Q_{g}) to the volume of liquid (Qₗ) passing through the nozzle is in the range 0.5:1 to 50:1, preferably 1:1 to 10:1 (where the ratio of the volume of gas (Q_{g}) to the volume of liquid (Qₗ) is determined at the desired reaction temperature and pressure).

14. A process as claimed in any one of the preceding claims wherein the shearing forces exerted on the suspension in the high shear mixing zone(s) are sufficiently high that at least a portion of the synthesis gas is broken down into gas bubbles having diameters in the range of from 1 μm to 10 mm, preferably from 30 μm to 3000 μu, more preferably from 30 μm to 300 μm.

15. A process as claimed in any one of the preceding claims wherein the kinetic energy dissipation rate in the high shear mixing zone(s) is in the range of from 0.5 to 25 kW/m³, relative to the total volume of suspension present in the system, more preferably from 0.5 to 10 kW/m³, most preferably from 0.5 to 5 kW/m³, and in particular, from 0.5 to 2.5 kW/m³.

16. A process as claimed in any one of the preceding claims wherein the suspension recycle stream is withdrawn from the reactor vessel and is at least in part recycled to a high shear mixing zone(s) through an external conduit having a mechanical pumping means positioned therein and the suspension recycle stream is cooled by means of a heat exchanger positioned on the external conduit.

17. A process as claimed in claim 16 wherein the ratio of the volume of the external conduit (excluding the volume of the external heat exchanger) to the volume of the reactor vessel is in the range of 0.005:1 to 0.2:1.

18. A process as claimed in any one of the preceding claims wherein a vaporizable coolant liquid is introduced into the reactor system.

19. A process as claimed in any one of the preceding claims wherein a gas cap containing a gaseous phase comprising unconverted synthesis gas, carbon dioxide, inert gases such as nitrogen, gaseous hydrocarbons, vaporized low boiling liquid hydrocarbons, vaporized water by-product and any vaporized liquid coolant is present in the top of reactor vessel above the level of suspension and a gaseous stream is recycled from the gas cap to the high shear mixing zone(s).

20. A process as claimed in any one of the preceding claims wherein the average residence time of the liquid component of the suspension in the system is in the range from 10 minutes to 50 hours, preferably 1 hour to 30 hours.

21. A process as claimed in any one of the preceding claims wherein the system is operated with a gas hourly space velocity (GHSV) in the range 100 to 40000 h⁻¹, more preferably 1000 to 30000 h⁻¹, most preferably 2000 to 15000 h⁻¹, for example, 4000 to 10000 h⁻¹ at normal temperature and pressure (NTP) based on the feed volume of synthesis gas at NTP.

22. A process as claimed in any one of the preceding claims wherein the catalyst is cobalt on zinc oxide.

23. A process as claimed in any one of the preceding claims wherein the catalyst has a particle size in the range 5 to 500 microns, more preferably 5 to 100 microns, most preferably, in the range 5 to 30 microns.

24. A process as claimed in any one of the preceding claims wherein the suspension of catalyst discharged into the reactor vessel comprises less than 40% wt of catalyst particles, more preferably 10 to 30% wt of catalyst particles, most preferably 10 to 20% wt of catalyst particles.
